(19) United States
(12) Patent Application Publication
(10) Pub. No.: US 2024/0296960 A1
(43) Pub. Date: Sep. 5, 2024

(54) METHOD AND SYSTEM FOR MAPPING INDIVIDUALIZED METABOLIC PHENOTYPE TO A DATABASE IMAGE FOR OPTIMIZING CONTROL OF CHRONIC METABOLIC CONDITIONS

(71) Applicant: UNIVERSITY OF VIRGINIA PATENT FOUNDATION, Charlottesville, VA (US)

(72) Inventors: BORIS P. KOVATCHEV, Charlottesville, VA (US); MARC D. BRETON, Charlottesville, VA (US); LEON S. FARHI, Charlottesville, VA (US); JAGDISH THAKUR, Zion Crossroads, VA (US)

(21) Appl. No.: 18/278,485

(22) PCT Filed: Feb. 23, 2022
(86) PCT No.: PCT/US22/17449

§ 371 Date: Aug. 23, 2023

Related U.S. Application Data
(60) Provisional application No. 63/152,683, filed on Feb. 23, 2021.

(30) Foreign Application Priority Data
Feb. 23, 2021 (US) 63152683

(51) Int. Cl. G16H 50/50; G16H 50/70

(52) U.S. Cl. CPC G16H 50/50; G16H 50/70

(57) ABSTRACT

Provided are a method, system and computer-readable storage medium for mapping a metabolic phenotype of a subject to a database entity for creating a digital twin of the subject, and enabling as to such subject, one or more of in silico titration of treatment as to such subject prior to initiating clinical intervention, in silico adjustment and optimization of medication dosing and timing, personalized training through in silico replay of particularized treatment scenario, and tracking over time of any divergence in one or more metabolic traits as between the subject and the digital twin to thereby detect deterioration or improvement in the subject's condition.

### CROSS-REFERENCE TO RELATED APPLICATION

This international application claims priority to and the benefit of U.S. Provisional Application No. 63/152,683 filed Feb. 23, 2021, the entire contents of which are incorporated by reference herein.

### FIELD OF THE DISCLOSURE

Disclosed embodiments relate to glucose control, and more specifically, to such control as enabled by projection of a subject's metabolic phenotype onto an in silico entity, resulting in a simulated reproduction of such phenotype.

### BACKGROUND

In connection with discussion herein, superscript notations herein are to those references as delineated in the similarly entitled section herein.

Glucose dysregulation conditions, e.g., Type 1 and Type 2 diabetes, obesity, pre-diabetes, or gestational diabetes are among the best quantified human ailments.¹ Real-time signals such as self-monitoring of blood glucose (SMBG),²⁻⁵ or continuous glucose monitoring (CGM),⁶⁻⁸ are readily available and supported by a variety of metrics of glycemic control.⁹ Elaborate models describe the action of the metabolic system;¹⁰⁻¹⁴ and insulin delivery can be automated by artificial pancreas technologies,¹⁵⁻¹⁹ approved by the FDA for routine clinical use in Type 1 diabetes for controlling blood glucose fluctuations in a person's natural environment²⁰⁻²³. A number of medication treatments are available for people with Type 2 diabetes.²⁴⁻²⁷ In these regards, it would be advantageous to provide a manner of enabling a simulated forecast of an effect of proposed changes in diabetes treatment(s) prior to instituting such changes clinically.

### SUMMARY

It is to be understood that both the following summary and the detailed description are exemplary and explanatory and are intended to provide further explanation of the present embodiments as claimed. Neither the summary nor the description that follows is intended to define or limit the scope of the present embodiments to the particular features mentioned in the summary or in the description. Rather, the scope of the present embodiments is defined by the appended claims.

We therefore propose to advance beyond the established medical paradigms, and thus present treatment of glucose dysregulation as a multi-step process that is (a) initialized with each subject's demographic information, (b) periodically updated with data from electronic health records (EHRs), and (c) delivered by individualized algorithms. To enable this treatment paradigm change, we, at the University of Virginia (UVA) have created and tested a new quantitative method encompassing a Virtual Image of the Person (VIP), i.e., a “digital twin” particularly selected from a predefined database of in silico entities to match metabolic and demographic data typically available in a subject's EHR. In this regard, it will be assumed that each in silico entity is well characterized by one or more physiological features, e.g., fasting glucose, fasting insulin or c-peptide, metrics of beta-cell function of insulin resistance, 28-29 and/or metrics based on simulated SMBG or CGM traces.⁹ Such features are typically available in comprehensive in silico populations, in particular in UVA's metabolic simulation environment. As such, and in view of the above, it would be beneficial to provide and ensure optimization of the above-discussed VIP yielding various manner of treatment, education, and monitoring of glucose control scenarios.

Embodiments may include a method, system, computer-readable storage medium regarding obtaining one or more in vivo glucose-metabolism traits of a subject, comparing the one or more in vivo glucose-metabolism traits of the subject to a corresponding one or more glucose-metabolism traits of one or more in silico entities, and based on the comparing, determining at least one matching in silico entity for the one or more glucose-metabolism traits of the subject, and assigning in vivo behavioral and demographic characteristics for the subject to the at least one matching in silico entity.

The glucose-metabolism traits may include one or more of (a) hemoglobin A1c (HbA1c), (b) fasting glucose, (c) C-peptide, (d) HOMA2-B, (e) HOMA-IR, or (f) any combination thereof.

The behavioral and demographic characteristics may include one or more of (1) age of the subject, (m) duration of diabetes for the subject, (n) body mass index (BMI) of the subject, (o) body weight (BW) of the subject, or (p) any combination thereof.

The determining and/or the assigning may be performed, depending upon the availability of data therefor, in a single pass or iteratively.

The subject may be a human or an animal, relative to a number of corresponding images of an in silico entity therefor as stored in a database comprising a population of in silico entity images.

The determining at least one matching in silico entity for the one or more glucose-metabolism traits of the subject may be based on a least magnitude Euclidean distance evaluated for at least (x) the subject and the at least one matching in silico entity for the one or more glucose-metabolism traits of the subject and (y) the subject and another in silico entity for the one or more glucose-metabolism traits of the subject.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. Embodiments herein will be more particularly described in conjunction with the following drawings wherein:

FIG. 1 illustrates a manner of creating a digital twin of a subject for addressing glucose dysregulation according to embodiments herein;

FIGS. 2-5 illustrate matching for HbA1c relative to training and simulation for creating a digital twin of a subject for addressing glucose dysregulation according to embodiments herein;

FIGS. 6-25A illustrate matching for HbA1c, age, duration of diabetes, BMI, and BW in FIGS. 6-9A, FIGS. 10-13A, FIGS. 14-17A, FIGS. 18-21A, and FIGS. 22-25A, respectively, relative to training and simulation for creating a digital twin of a subject for addressing glucose dysregulation according to embodiments herein;

FIG. 26 illustrates a heat plot of correlation coefficient difference relative to validation of creation of the digital twin herein with respect to metabolic and demographic parameters;

FIGS. 27-46A illustrate matching for HbA1c, age, duration of diabetes, BMI, and BW in FIGS. 27-30A, FIGS. 31-34A, FIGS. 35-38A, FIGS. 39-42A, and FIGS. 43-46A, respectively, relative to testing and simulation for creating a digital twin of a subject for addressing glucose dysregulation according to embodiments herein; and

FIGS. 47-52 illustrate various apparatuses and environments for implementing a digital twin of a subject for addressing glucose dysregulation according to embodiments herein.

### DETAILED DESCRIPTION

The present disclosure will now be described in terms of various exemplary embodiments. This specification discloses one or more embodiments that incorporate features of the present embodiments. The embodiment(s) described, and references in the specification to “one embodiment,” “an embodiment,” “an example embodiment,” etc., indicate that the embodiment(s) described may include a particular feature, structure, or characteristic. Such phrases are not necessarily referring to the same embodiment. The skilled artisan will appreciate that a particular feature, structure, or characteristic described in connection with one embodiment is not necessarily limited to that embodiment but typically has relevance and applicability to one or more other embodiments.

In the several figures, like reference numerals may be used for like elements having like functions even in different drawings. The embodiments described, and their detailed construction and elements, are merely provided to assist in a comprehensive understanding of the present embodiments. Thus, it is apparent that the present embodiments can be carried out in a variety of ways, and does not require any of the specific features described herein. Also, well-known functions or constructions are not described in detail since they would obscure the present embodiments with unnecessary detail.

The description is not to be taken in a limiting sense, but is made merely for the purpose of illustrating the general principles of the present embodiments, since the scope of the present embodiments are best defined by the appended claims.

It should also be noted that in some alternative implementations, the blocks in a flowchart, the communications in a sequence-diagram, the states in a state-diagram, etc., may occur out of the orders illustrated in the figures. That is, the illustrated orders of the blocks/communications/states are not intended to be limiting. Rather, the illustrated blocks/communications/states may be reordered into any suitable order, and some of the blocks/communications/states could occur simultaneously.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A);

in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedure, Section 2111.03.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. The word “exemplary” is used herein to mean “serving as an example, instance, or illustration.” Any embodiment described herein as “exemplary” is not necessarily to be construed as preferred or advantageous over other embodiments. Additionally, all embodiments described herein should be considered exemplary unless otherwise stated.

It should be appreciated that any of the components or modules referred to with regards to any of the embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Ranges may be expressed herein as from “about” or “approximately” one particular value and/or to “about” or “approximately” another particular value. When such a range is expressed, other exemplary embodiments include from the one particular value and/or to the other particular value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., a rat, dog, pig, or monkey), etc. It should be appreciated that the subject may be any applicable human patient, for example.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Relative to the aforementioned implementation and verification as regards a Virtual Image of a Person (VIP), disclosed is a system and method for iterative mapping of subject's metabolic phenotype to an in silico entity, i.e., a VIP defining a “digital twin” selected from a predefined database of in silico entities, each characterized by one or more physiological features, e.g., fasting glucose, fasting insulin or c-peptide, metrics of beta-cell function of insulin resistance, blood glucose self-monitoring (SMBG) or continuous glucose monitoring (CGM) traces. As noted previously, the term “subject” may be inclusive of either a human or an animal. As such, where the term “VIP” is stated, it is to be understood that the same may be in regard to a Virtual Image of a Subject, or “VIS.” In these ways, references to the term VIP may be interchangeable with a Virtual Image of an Animal, or “VIA,” for which comparison may be made against a corresponding animal according to the method herein. The method herein consists of two steps: (i) observed in vivo glucose-metabolism traits of a subject are mapped to an in silico entity with similar physiological features (i.e., forward in-vivo to in silico mapping), and (ii) behavioral and demographic characteristics observed in vivo are transferred/assigned to that corresponding in silico entity (i.e., in-vivo to in silico backpropagation of data). When these two steps are accomplished, each subject is mirrored to a metabolic and demographic “digital twin,” which enables computer simulation experiments for the purpose of: (i) in silico titration of treatment to each subject prior to initiating clinical intervention; (ii) in silico adjustment and optimization of medication dosing and timing for the subject; (iii) subject training through in silico replay of their own treatment scenarios (which can be made available to both subjects and health care providers), and (iv) tracking over time of any divergence between in vivo and in silico traits to thereby detect deterioration or improvement in a subject's condition, which may be quantified as deviation from their “digital twin.”

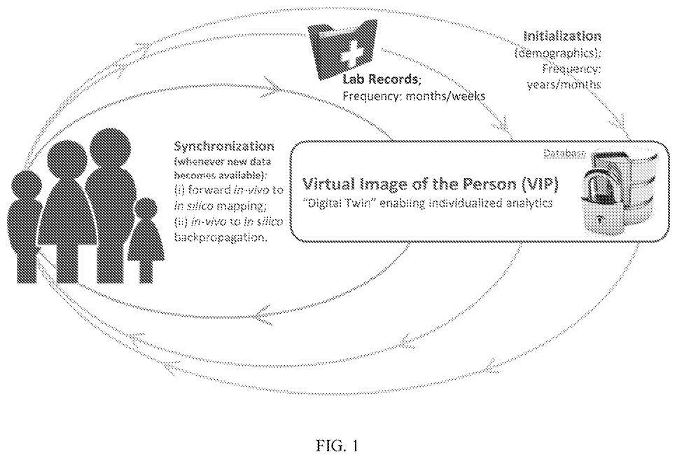

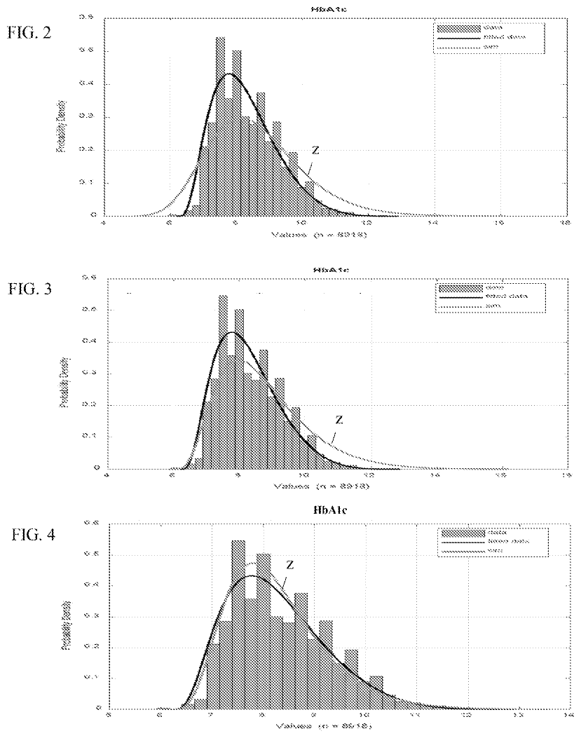

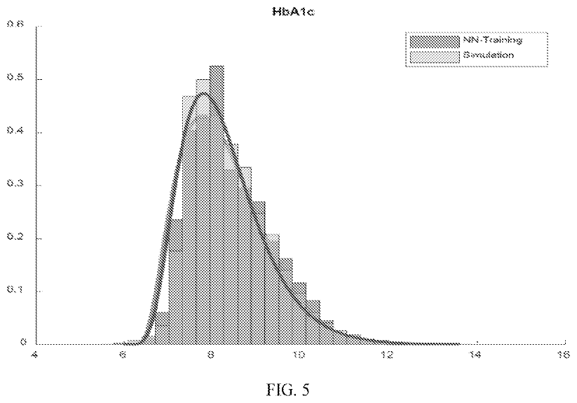

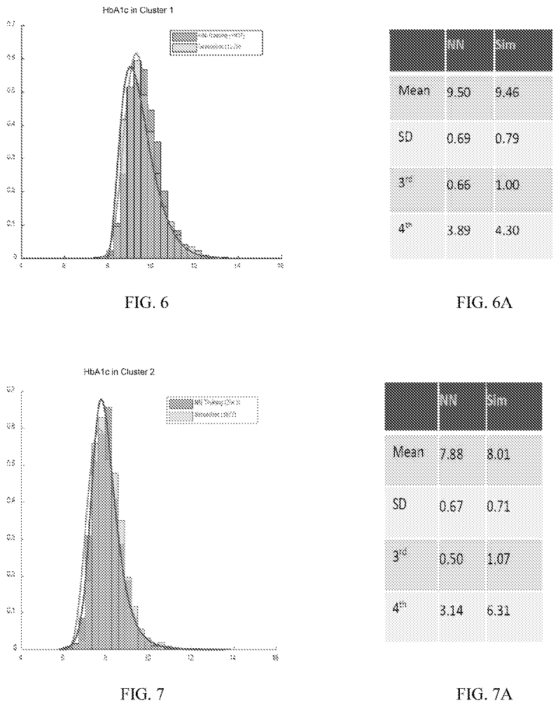

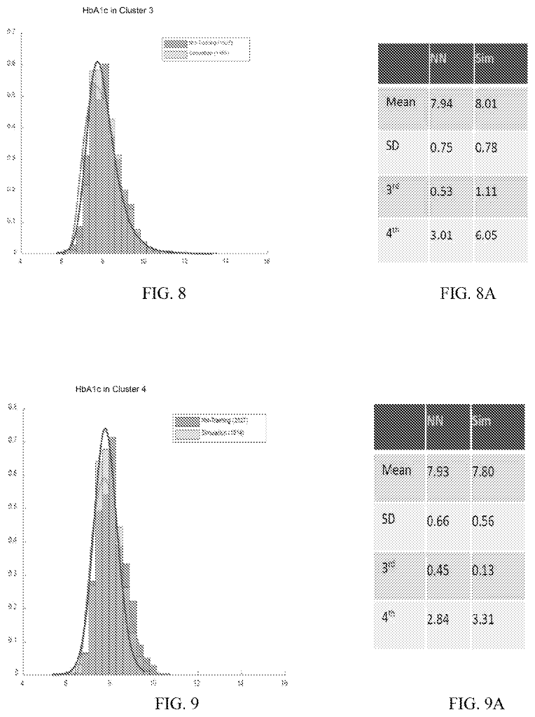

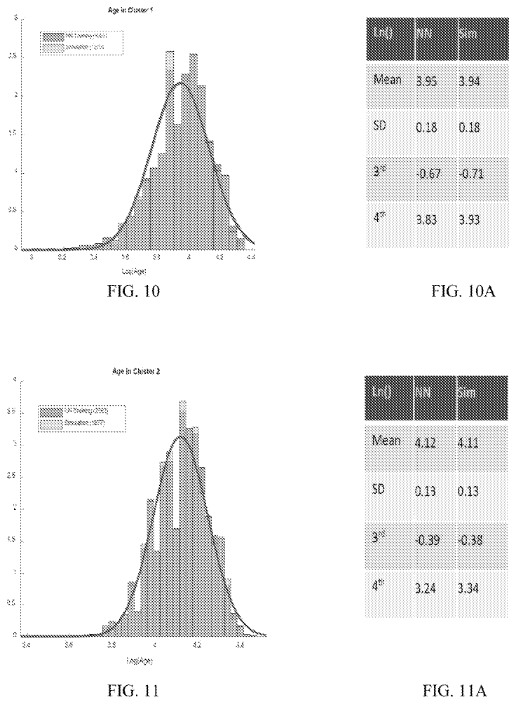

In referring to FIG. 1, there is shown a generalized explanation of creation of the VIP via the aforementioned mapping and backpropagation, in which data from different sources are combined within a VIP, the creation of which forms a database vault that contains individual information for a subject. The information is structured to reflect different time frames (e.g., months/weeks) so as to iteratively synchronize disparate data sets.

Therein, shown are signals and time frames for the iterative mapping of a subject's metabolic phenotype to an in silico entity—a VIP defining a “digital twin.” As shown, the method consists of the above-noted two steps, including (i) observed in vivo glucose-metabolism traits of a subject are mapped to an in silico entity with similar physiological features (forward in vivo to in silico mapping), and (ii) behavioral and demographic characteristics observed in vivo are transferred/assigned to the corresponding in silico entity (in-vivo to in silico backpropagation of data). When these two steps are accomplished, the subject and in silico populations are synchronized and each subject is mirrored by a digital twin. The accomplishment of these two steps may be achieved in one pass, i.e., in a given cycle, in a case where all of the necessary data is available. Alternatively, the accomplishment of these two steps may be achieved iteratively, such that one or more of the steps may be performed repetitively (until completion of all steps) depending upon the availability of data necessary for that performance.

The discussion below provides an outline for embodiments herein in which empirical matching of certain physio-behavioral parameters allows for certain physio-behavioral parameters, e.g., Hemoglobin A1c (HbA1c), to be matched between a subject and an in silico entity by iterative replay of simulation scenarios. This is because the values of HbA1c are influenced by many factors such as behavioral interventions (meals or exercise) and treatments that can be replayed in silico.

Mapping a real subject or a group of subjects to in silico entities is then a two-step procedure as delineated below:

(1) Forward in-vivo to in silico mapping: The procedure assumes that a virtual population is available, in which a multitude of in silico entities are defined by one or more physiologic parameters, such as fasting glucose, C-peptide, HOMA2B, HOMA2IR, which correspond to similar metabolic characteristics of the subject; thus, the subject can have one or more physiologic parameters in common with and available for one or more in silico entities. As such, the method begins a search for the in silico entity that is a closest match to each real subject according to one or more of the parameters; and

(2) In-vivo to in silico backpropagation of data: This procedure is performed for the purpose of then equipping each mapped in silico entity with corresponding subject demographics, such as ages, duration of diabetes, body mass index (BMI), or other characteristics (e.g. body weight (BW)) typically unavailable in silico. The assumption is that such data is available for a sufficiently large subject population, when considered together with physiological traits that are common within the in silico population (e.g., fasting glucose, fasting C-peptide, HOMA2-B, HOMA2-IR, HbA1c).

Data: Dataset of 17092 records (all for Type 2 diabetes persons). These subjects had available demographic and metabolic data including age, duration of diabetes, body weight, body mass index (BMI), fasting glucose and fasting c-peptide, as well as their derivative assessments of beta-cell function and insulin resistance, HOMA2-B and HOMA2-IR.²⁹ These subjects were stratified into four (4) clinically identifiable subtypes (clusters) of diabetes: SIDD (severe insulin-deficient diabetes), SIRD (severe insulin-resistant diabetes), MOD (mild obesity-related diabetes), and MARD (mild age-related diabetes).

Available in silico population: An in silico population of N=6156 in silico entities has been established in previous studies, matching the distributions across four (4) clinical subtypes of Type 2 diabetes, of several observable physiological variables, including, but not limited to, fasting glucose, C-peptide, HOMA2B, HOMA2IR.

Testing and Validation: The data was randomly assigned to two groups: Training Data Set (N=8924) (NN-Training in the figures) and Test Data Set (N=8922) (NN-Testing in the figures). The Training Data Set was used to develop the VIP via matching of the physiological and demographic variables distributions and cluster memberships between in silico entities and real subjects, after which the VIP was fixed.

To validate the VIP on independent data, after fixing the mapping procedure in the Training Data, the “Digital Twin” was applied to the Test Data set and judged for its ability to: (i) map persons to in silico entities, i.e., to their “digital twins,” and (ii) backpropagate demographic characteristics from the Test population to the in silico cohort, thus accurately preserving the known clinical subtypes (clusters).

Applications: As a result of the method herein, each subject can mirror a metabolic/demographic “digital twin” which can enable computer simulation experiments for the purpose of: (i) in silico titration of treatment to each subject prior to initiating clinical intervention; (ii) in silico adjustment and optimization of medication dosing and timing; (iii) subject training through in silico replay of their own treatment scenarios, available to both subjects and health care providers, and (iv) tracking over time any divergence between in vivo and in silico traits and thereby detecting deterioration or improvement in a subject's condition quantified as deviation from their “digital twin.”

In addition to Type 2 diabetes, the method is applicable without modification to obesity, pre-diabetes, gestational diabetes, or other glucose metabolic disorders. Further implementation with real-time signals is possible as well, and is primarily reserved for real-time applications, such as an artificial pancreas (AP).

Creation of the VIP, as the “digital twin,” begins with initial training therefor encompassing forwarding in vivo to in silico mapping and in vivo to in silico backpropagation of data. The training is described below, based on a virtual population of in silico entities.

### Forwarding In Vivo to in Silico Mapping

Such forwarding may include that of physio-behavioral parameters, such as HbA1c, and other metabolic characteristics so as to assist in obtaining a digital match for a subject.

In these regards, values of Hemoglobin A1c (HbA1c) are influenced by many factors such as behavioral interventions (meals or exercise) and treatments.

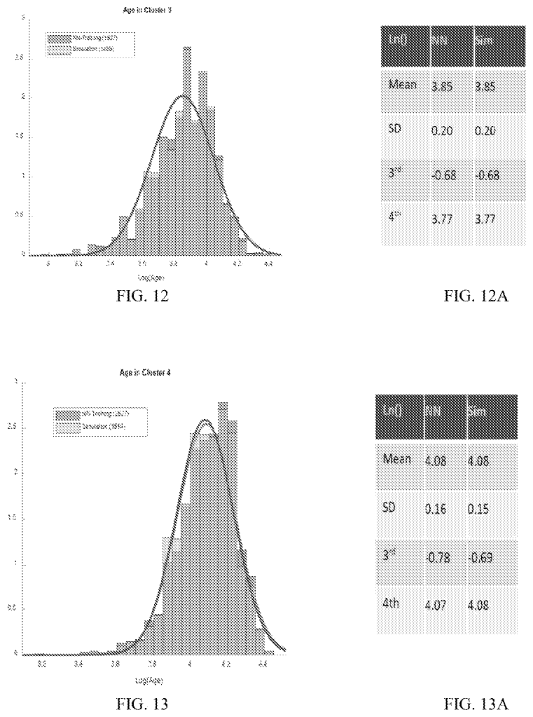

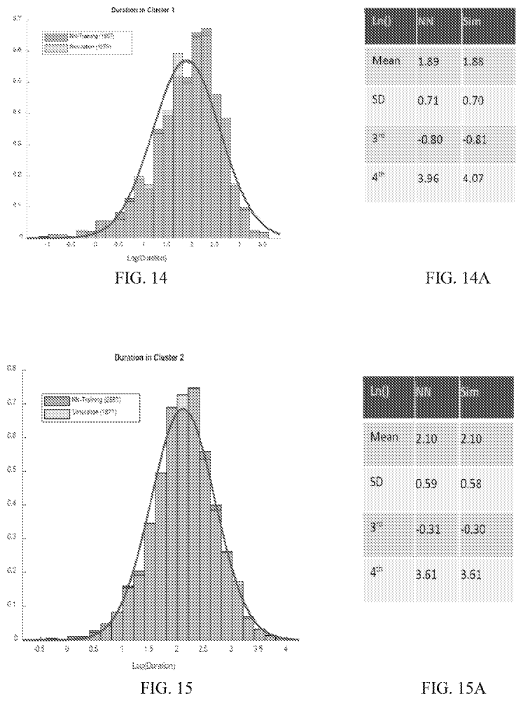

FIGS. 2-4 show matching for HbA1c relative to the training set (N=8918 after removing six entities with fasting glucose <70 mg/dL), and the simulation (z) excluding subjects having glucose <70 or >500 mg/dL. With particular reference to FIG. 2, three-meals were administered during a one-day simulation (0600-0600) at 0700/1200/1800 respectively according to sizes of (0.4/0.3/0.3)/2 g/BW (body weight in kg.). In referring to FIG. 3, meal size and frequency were increased to match the left tail (HbA1c<7.0), whereas mealtimes consisted of 0700/1200/1700/2000 with respective sizing of 1.4((0.4/0.3/0.3/0.3)/2 g/BW. Right-tail matching (HbA1c>7.5) was, as is shown in FIG. 4, achieved according to a standardized transform. FIG. 5 illustrates a resultant population-level distribution for HbA1c. FIGS. 6A-9B illustrate a breakdown (mean, standard deviation (SD), skewness (3rd), and kurtosis (4th), where Ln represents taking the natural logarithm of the values) for HbA1c relative to the training set versus simulation for diabetes clustered subtypes including SIDD (severe insulin-deficient diabetes), SIRD (severe insulin-resistant diabetes), MOD (mild obesity-related diabetes), and MARD (mild age-related diabetes).

Forwarding other than HbA1c relative to the in silico population may be achieved via computation of the metabolic fluxes within such population. As such, the procedure assumes that certain metabolic characteristics of the subject (e.g., fasting glucose, C-peptide, HOMA2B, HOMA2IR) are available, or can be simulated, for each in silico entity; thus, the subject can have one or more physiologic parameters in common and available within the in silico population. As such, the method begins a search for the in silico entity that is a closest match to each real subject, as follows:
a. A distance matrix (D_{n×m}) is defined consisting of weighted Euclidean distances between all in silico entities and subjects. The distances are computed for vectors consisting of the available common parameters (e.g., fasting glucose, fasting C-peptide, HOMA2-B, HOMA2-IR, HbA1c);
b. Each element of the distance matrix uses the following formula: D_{n×m}=dᵢⱼ{i=1, . . . , n; j=1, . . . , m}, where n is the number of entities in the in silico population and m≤1 is number of subjects to be mapped to in silico entities;
c. The distance, dᵢⱼ, between an in silico entity i and a subject j is then defined as the square root of the sum of squared differences between each weighted common variable Vₖ:

$${d}_{i j} = {( {\sum }_{k 1}^{6} {( {V}_{k}^{S i l i c o} ( i ) - {V}_{k}^{T r a i n} ( j ) )}^{2} )}^{1 / 2}$$

where Vₖ is the k^{th} weighted variable defined as Vₖ=k^{th} variable/k^{th} variable SD.

Therefore, the distance matrix consists of the weighted distances between all possible in silico-subject pairs in the common variable space. The global minimum of all elements of the distance matrix (D_{n×m}), i.e., the least magnitude Euclidean distance, identifies the best matched (i.e., closest) entity for an in silico-subject pair.
d. Once a matching pair is identified, the corresponding records are removed from the distance matrix, thereby reducing its dimensions by one (1). A new search is then performed for the next global minimum in the reduced matrix until another matching pair is identified. The process continues iteratively until all subjects are matched to in silico entities.

### In Vivo to in Silico Backpropagation of Data

Such backpropagation further tailors the “digital twin” by imparting demographic characteristics from the subject which are typically unavailable in the in silico population. Accordingly, the procedure is performed to the purpose of equipping each in silico entity with “subject” demographics, such as ages, duration of diabetes, BW, and BMI. The assumption is that such data are available for a sufficiently large subject population, together with physiological traits that are common with the in silico population (e.g., fasting glucose, fasting C-peptide, HOMA2-B, HOMA2-IR, HbA1c). The procedure then works as follows:
a. In silico-subject pairing is performed as described above;
b. For each in silico-subject pair, demographic variables (e.g., age at diagnosis, duration of diabetes, BMI, or other) are extracted from the subject's data record and assigned to the matching in silico entity;
c. At the end of the procedure, each virtual entity in the in silico population is equipped with a complete set of both physiologic and demographic parameters.

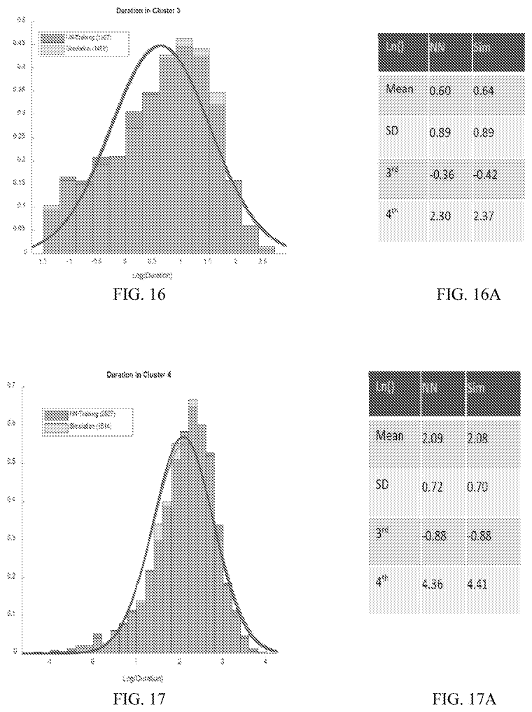

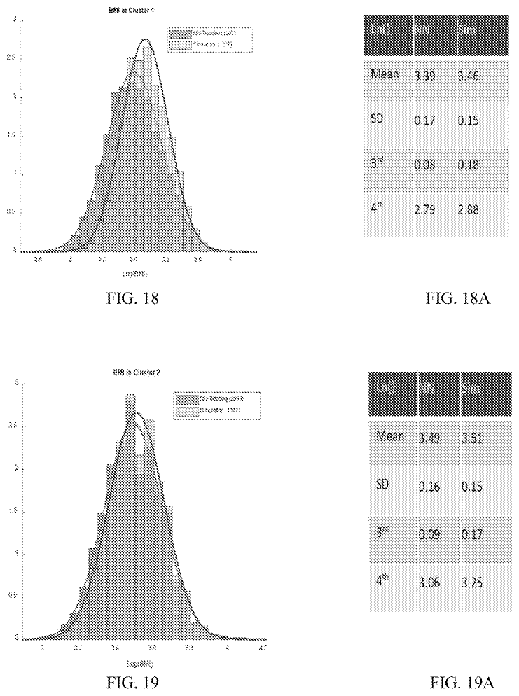

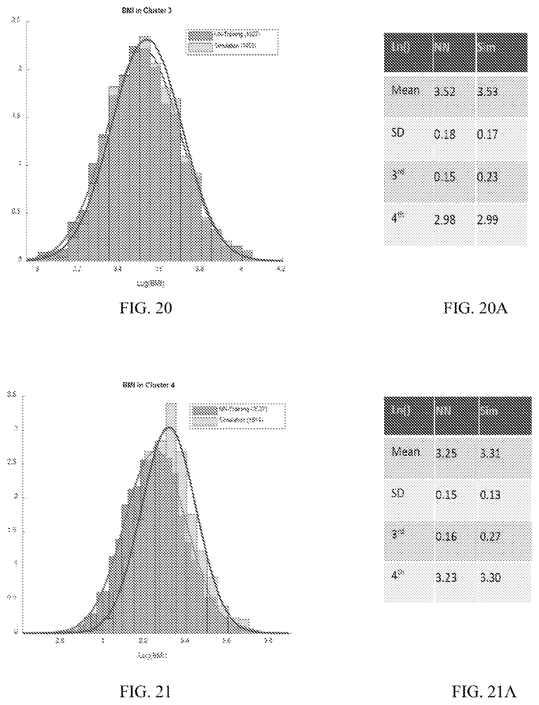

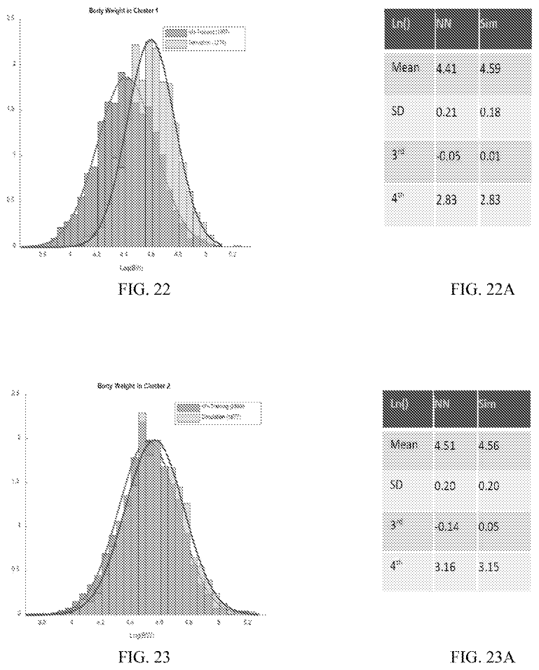

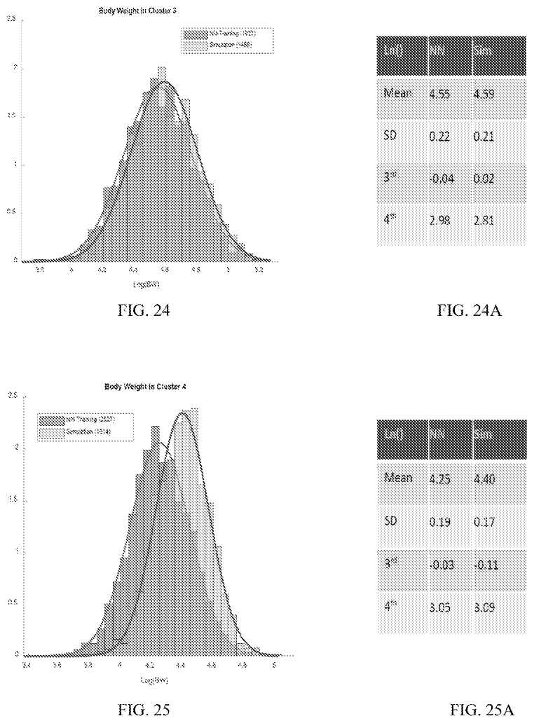

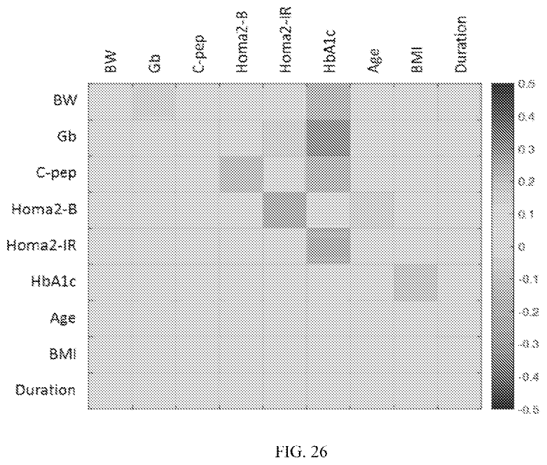

Results based on the training data set are illustrated in FIGS. 10-25A. More specifically, comparisons (training versus simulation) are shown for age, duration of diabetes, BMI, and BW in FIGS. 10-13A, FIGS. 14-17A, FIGS. 18-21A, and FIGS. 22-25A, respectively.

Validation of the “Digital Twin” on Test Data

A. Classification into Clinical Subtypes (Clusters)

After fixing the mapping procedure in the Training Data, the “Digital Twin” method was applied to the Test Data set and judged for its ability to: (i) map subjects to in silico entities, i.e., to their “digital twins,” and (ii) backpropagate demographic characteristics from the Test population to the in silico cohort, while accurately preserving the known clinical diabetes subtypes. Table 1 below shows results for the in silico and testing populations by subtype (cluster).

TABLE 1
Memberships in virtual population and test data.

| In silico data (6156) | | | NN Test data (8922) | |
|---|---|---|---|---|
| Cluster | Number | % of the total | number | % of the total |
| 1 | 1311 | 21.30 | 1907 | 21.37 |
| 2 | 1719 | 27.92 | 2563 | 28.73 |
| 3 | 1283 | 20.84 | 1926 | 21.59 |
| 4 | 1843 | 29.94 | 2526 | 28.31 |

### B. Correlation Between Physiological and Demographic Variables

The correlation matrix of test set subjects and simulated entities are respectively given in Tables 2 and 3 below, with respect to BW, Gb, C-pep, HOMA2-B, HOMA2-IR, HbA1c, Age, BMI, and Duration of diabetes. A heat plot of correlation coefficient difference is shown in FIG. 26.

TABLE 2
Correlation matrix of test set

| | [symbol] | [symbol] | [symbol] | [symbol] | [symbol] | [symbol] | [symbol] | [symbol] | Duration |
|---|---|---|---|---|---|---|---|---|---|
| BW | 1 | 0.0854 | 0.4664 | 0.2629 | 0.4456 | −0.0372 | −0.1936 | 0.8523 | −0.1456 |
| Gb | | 1 | 0.0928 | −0.5758 | 0.3201 | 0.5768 | −0.0538 | 0.0469 | 0.0848 |
| C-pep | | | 1 | 0.6062 | 0.9489 | −0.0532 | −0.0558 | 0.4733 | −0.1626 |
| HOMA2-B | | | | 1 | 0.4253 | −0.3757 | −0.0331 | 0.2993 | −0.1670 |
| HOMA2-IR | | | | | 1 | 0.0690 | −0.0639 | 0.4427 | −0.1337 |
| HbA1c | | | | | | 1 | −0.1098 | −0.0307 | 0.0733 |
| Age | | | | | | | 1 | −0.1709 | 0.3533 |
| BMI | | | | | | | | 1 | −0.1510 |
| Duration | | | | | | | | | 1 |

[symbol]

indicates data missing or illegible when filed

TABLE 3
Correlation matrix of simulated subjects.

| | | | | | HOMA2- | | | | |
|---|---|---|---|---|---|---|---|---|---|
| | BW | Gb | C-pep | HOMA2-B | IR | HbA1c | Age | BMI | Duration |
| BW | 1 | 0.1208 | 0.3920 | 0.2042 | 0.4080 | 0.1004 | −0.1916 | 0.8248 | −0.1157 |
| Gb | | 1 | −0.0036 | −0.5858 | 0.1616 | 0.8896 | −0.1042 | 0.0617 | 0.0910 |
| C-pep | | | 1 | 0.7232 | 0.9799 | 0.0847 | −0.0464 | 0.4194 | −0.1629 |
| HOMA2-B | | | | 1 | 0.6051 | −0.4257 | 0.0177 | 0.2718 | −0.1738 |
| HOMA2-IR | | | | | 1 | 0.2320 | −0.0639 | 0.4243 | −0.1448 |
| HbA1c | | | | | | 1 | −0.1183 | 0.0633 | 0.0630 |
| Age | | | | | | | 1 | −0.1652 | 0.3426 |
| BMI | | | | | | | | 1 | −0.1341 |
| Duration | | | | | | | | | 1 |

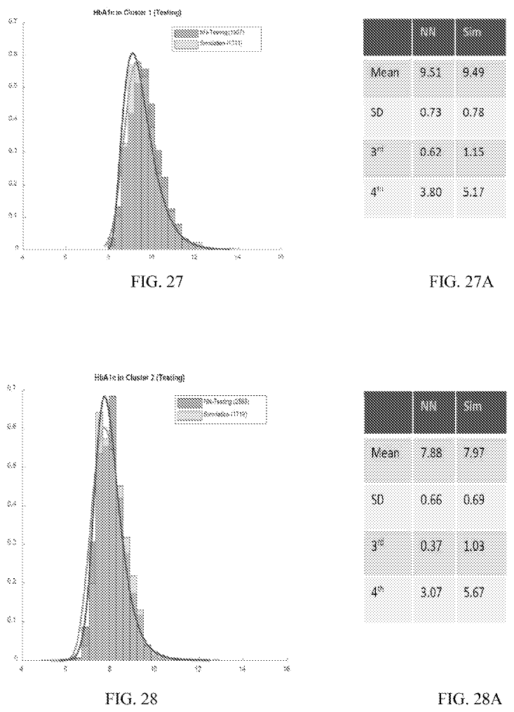

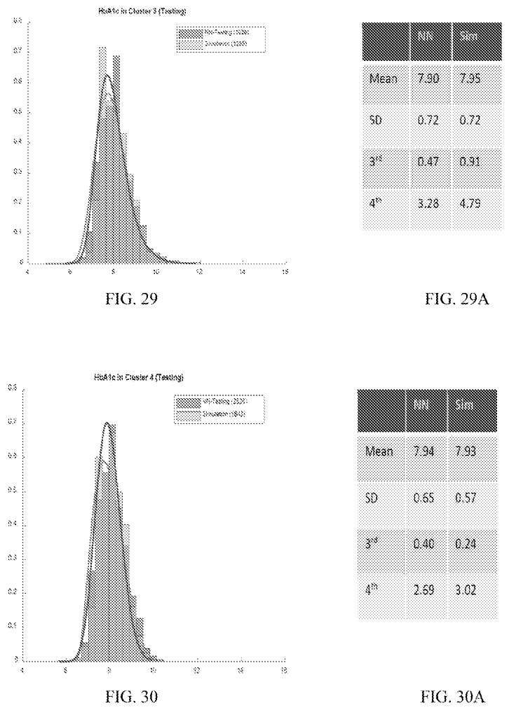

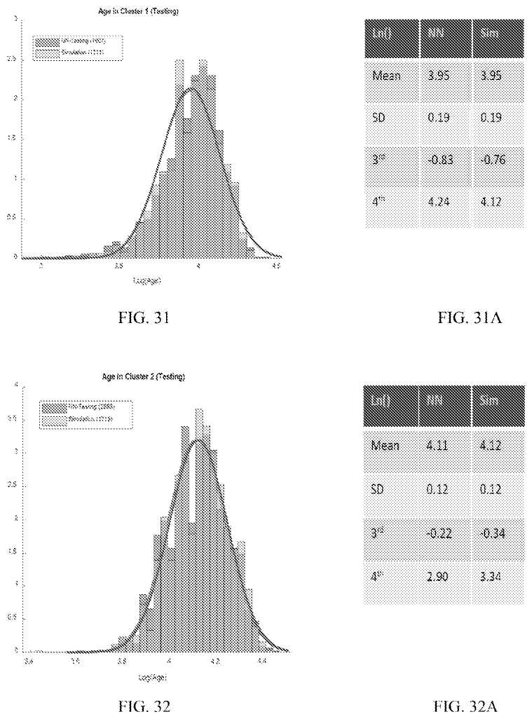

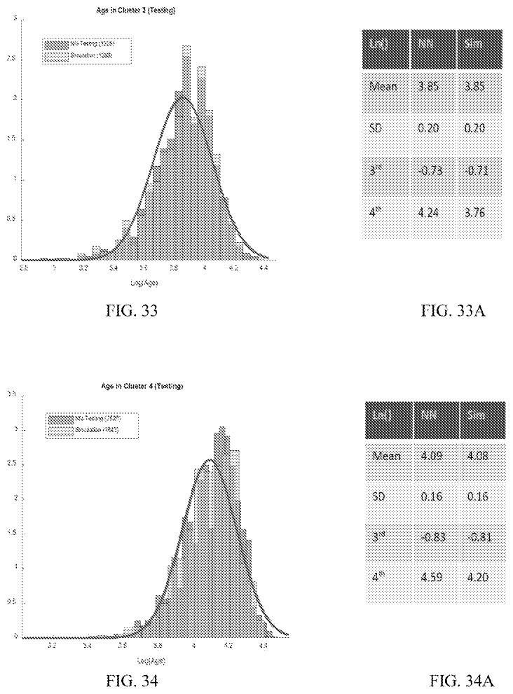

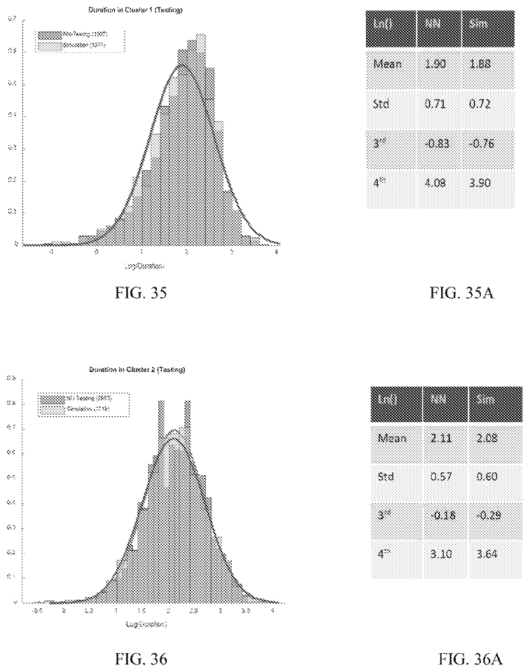

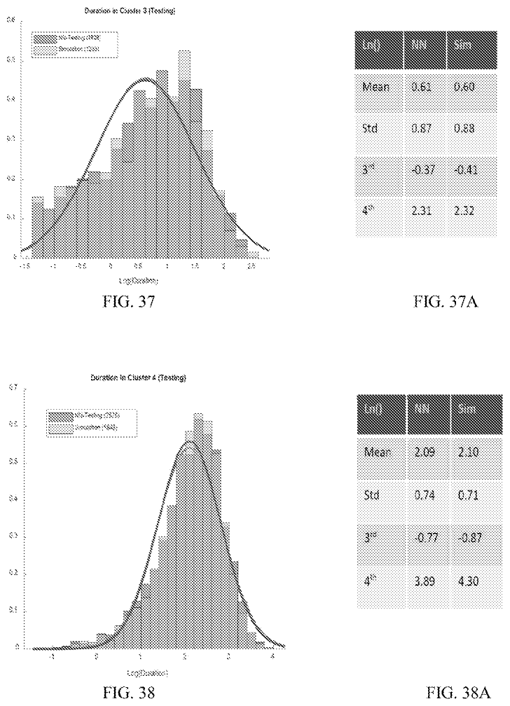

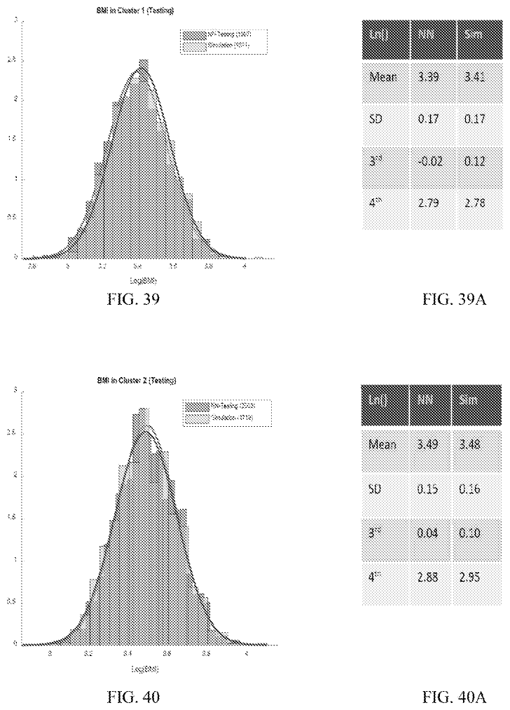

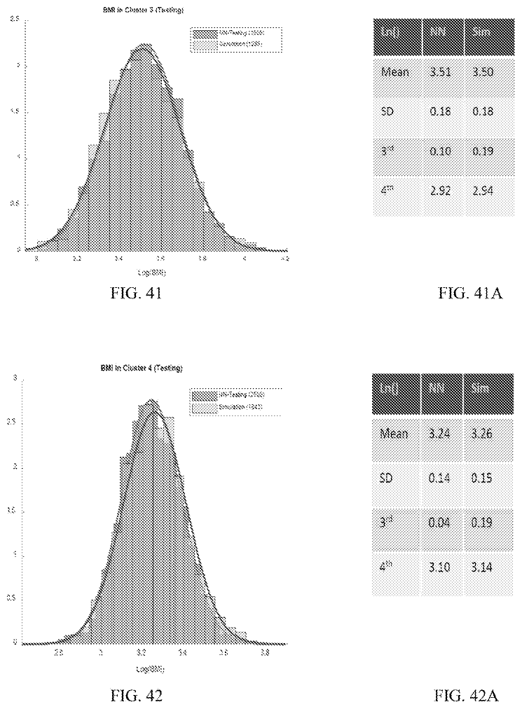

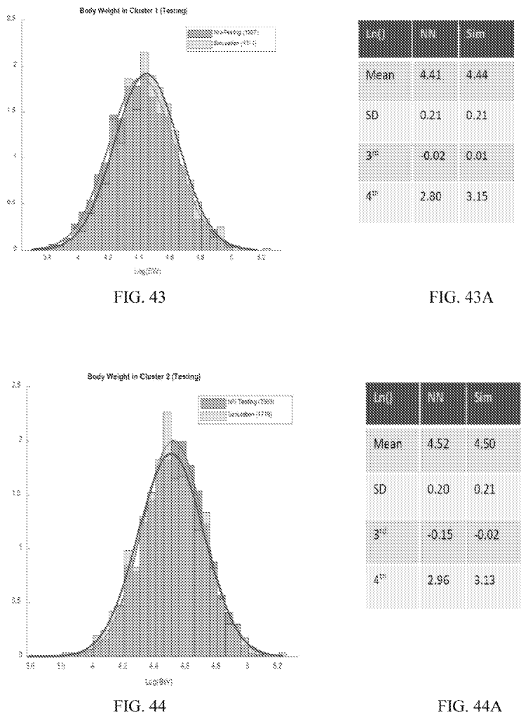

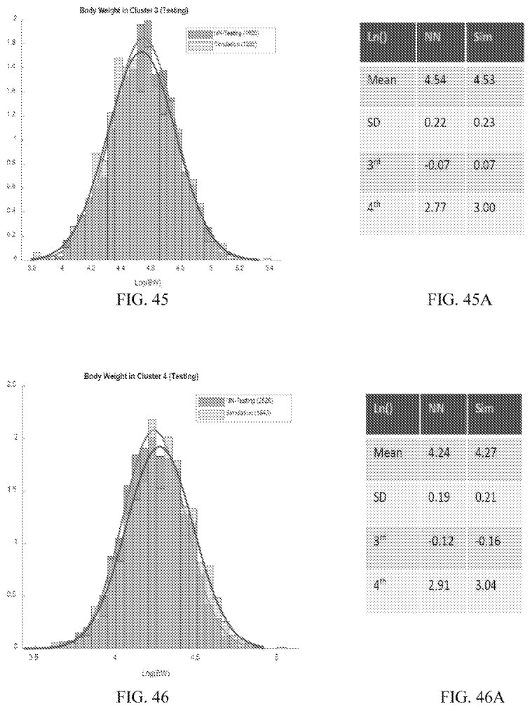

Similar to comparison of operation of the method as against the training data with respect to at least HbA1c, age, duration of diabetes, BMI, and body weight (as shown in FIGS. 6-25A), validation of the method herein further encompasses performing similar comparisons with respect to these variables as against the testing data, as is shown in FIGS. 27-46A. Accordingly, one or more aspects of the validation of the method may include (a) substantially maintaining cluster memberships (see Table 1 above), (b) substantially maintaining exemplary correlation(s) similar to those shown in the examples of above Tables 2 and 3, as well as (c) obtaining the aforementioned testing data comparisons showing substantially similar results to those shown by the exemplary, substantially matching distributions of FIGS. 27-46A as between the testing data and the data generated by the method.

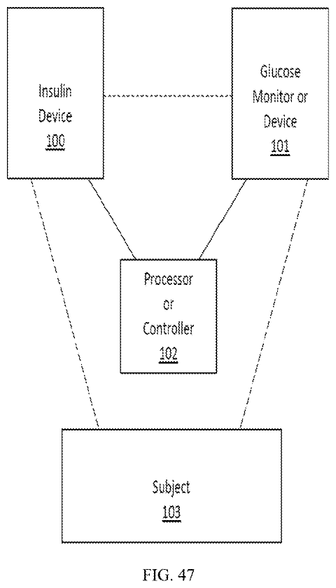

With respect to FIGS. 47-52, and when referring to FIG. 47, there is shown a high level functional block diagram of an artificial pancreas (AP) by which one or more aspects of the “digital twin” may be implemented and/or coordinated according to embodiments herein.

As shown, a processor or controller 102, may be configured to implement each of the prediction module and insulin infusion control module discussed above and to communicate with a CGM 101, and optionally with an insulin device 100 enabled to deliver insulin. The glucose monitor or device 101 may communicate with a subject 103 to monitor glucose levels thereof. The processor or controller 102 may be configured to include all necessary hardware and/or software necessary to perform the required instructions to achieve the aforementioned tasks. Optionally, the insulin device 100 may communicate with the subject 103 to deliver insulin thereto. The glucose monitor 101 and the insulin device 100 may be implemented as separate devices or as a single device in combination. The processor 102 may be implemented locally in the glucose monitor 101, the insulin device 100, or as a standalone device (or in any combination of two or more of the glucose monitor, insulin device, or a standalone device). The processor 102 or a portion of the AP may be located remotely, such that the AP may be operated as a telemedicine device.

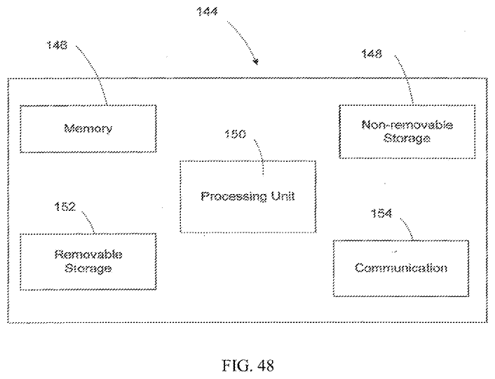

Referring to FIG. 48, a computing device 144 may implement the AP and may typically include at least one processing unit 150 and memory 146. Depending on the exact configuration and type of computing device, memory 146 may be volatile (such as RAM), non-volatile (such as ROM, flash memory, etc.) or some combination of the two.

Additionally, computing device 144 may also have other features and/or functionality. For example, the device could also include additional removable and/or non-removable storage including, but not limited to, magnetic or optical disks or tape, as well as writable electrical storage media. Such additional storage may be represented as removable storage 152 and non-removable storage 148. Computer storage media may include volatile and nonvolatile, removable and non-removable media implemented in any method or technology for storage of information such as computer readable instructions, data structures, program modules or other data. The memory, the removable storage and the non-removable storage may comprise examples of computer storage media. Computer storage media may include, but not be limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, one or more components of the AP. One or more of memory 146, non-removable storage 148, and removable storage may be cooperable to store a database comprising the in silico population discussed herein.

The computer device 144 may also contain one or more communications connections 154 that allow the device to communicate with other devices (e.g. other computing devices). The communications connections may carry information in a communication media. Communication media may typically embody computer readable instructions, data structures, program modules or other data in a modulated data signal, such as a carrier wave or other transport mechanism and includes any information delivery media. The term “modulated data signal” may include a signal that has one or more of its characteristics set or changed in such a manner as to encode, execute, or process information in the signal. By way of example, and not limitation, communication medium may include wired media such as a wired network or direct-wired connection, and wireless media such as radio, RF, infrared and other wireless media. As discussed above, the term computer readable media as used herein may include both storage media and communication media.

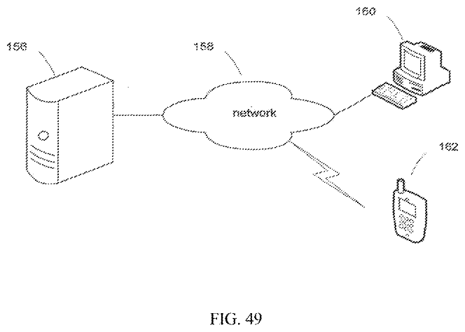

In addition to a stand-alone computing machine, embodiments herein may also be implemented on a network system comprising a plurality of computing devices that may in communication via a network, such as a network with an infrastructure or an ad hoc network. The network connection may include wired connections or wireless connections. For example, FIG. 49 illustrates a network system in which embodiments herein may be implemented. In this example, the network system may comprise a computer 156 (e.g., a network server), network connection means 158 (e.g., wired and/or wireless connections), a computer terminal 160, and a PDA (e.g., a smartphone) 162 (or other handheld or portable device, such as a cell phone, laptop computer, tablet computer, GPS receiver, mp3 player, handheld video player, pocket projector, etc. or other handheld devices (or non-portable devices) with combinations of such features). In an embodiment, it should be appreciated that the module listed as 156 may implement a CGM. In an embodiment, it should be appreciated that the module listed as 156 may be a glucose monitor device, an artificial pancreas, and/or an insulin device. Any of the components shown or discussed in FIG. 49 may be multiple in number. Embodiments herein may be implemented in anyone of the aforementioned devices. For example, execution of the instructions or other desired processing may be performed on the same computing device that is anyone of 156, 160, and 162. Alternatively, an embodiment may be performed on different computing devices of the network system. For example, certain desired or required processing or execution may be performed on one of the computing devices of the network (e.g. server 156 and/or a CGM), whereas other processing and execution of the instruction can be performed at another computing device (e.g., terminal 160) of the network system, or vice versa. In fact, certain processing or execution may be performed at one computing device (e.g. server 156 and/or insulin device, artificial pancreas, or CGM); and the other processing or execution of the instructions may be performed at different computing devices that may or may not be networked. For example, such certain processing may be performed at terminal 160, while the other processing or instructions may be passed to device 162 where the instructions may be executed. This scenario may be of particular value especially when the PDA 162 device, for example, accesses the network through computer terminal 160 (or an access point in an ad hoc network). For another example, software comprising the instructions may be executed, encoded or processed according to one or more embodiments herein. The processed, encoded or executed instructions may then be distributed to customers in the form of a storage media (e.g. disk) or electronic copy.

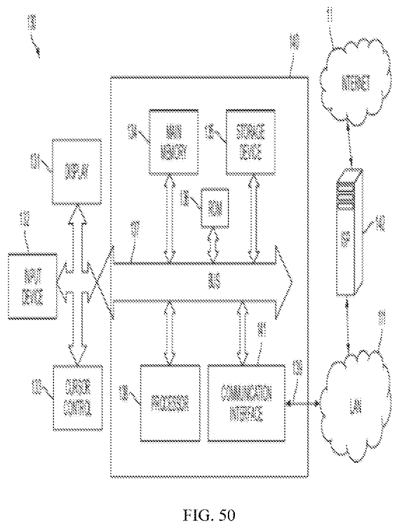

FIG. 50 illustrates a block diagram that of a system 130 including a computer system 140 and the associated Internet 11 connection upon which an embodiment may be implemented. Such configuration may typically used for computers (i.e., hosts) connected to the Internet 11 and executing software on a server or a client (or a combination thereof). A source computer such as laptop, an ultimate destination computer and relay servers, for example, as well as any computer or processor described herein, may use the computer system configuration and the Internet connection shown in FIG. 50. The system 140 may take the form of a portable electronic device such as a notebook/laptop computer, a media player (e.g., a MP3 based or video player), a cellular phone, a Personal Digital Assistant (PDA), a CGM, an AP, an insulin delivery device, an image processing device (e.g., a digital camera or video recorder), and/or any other handheld computing devices, or a combination of any of these devices. Note that while FIG. 48 illustrates various components of a computer system, it is not intended to represent any particular architecture or manner of interconnecting the components; as such, details of such interconnection are omitted. It will also be appreciated that network computers, handheld computers, cell phones and other data processing systems which have fewer components or perhaps more components may also be used. The computer system of FIG. 48 may, for example, be an Apple Macintosh computer or Power Book, or an IBM compatible PC. Computer system 140 may include a bus 137, an interconnect, or other communication mechanism for communicating information, and a processor 138, commonly in the form of an integrated circuit, coupled with bus 137 for processing information and for executing the computer executable instructions. Computer system 140 may also include a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 may further include a Read Only Memory (ROM) 136 (or other non-volatile memory) or other static storage device coupled to bus 137 for storing static information and instructions for processing by processor 138. A storage device 135, such as a magnetic disk or optical disk, a hard disk drive for reading from and writing to a hard disk, a magnetic disk drive for reading from and writing to a magnetic disk, and/or an optical disk drive (such as a DVD) for reading from and writing to a removable optical disk, may be coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer readable media may provide non-volatile storage of computer readable instructions, data structures, program modules and other data for the general purpose computing devices. Typically, computer system 140 may include an Operating System (OS) stored in a non-volatile storage for managing the computer resources and may provide the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of OSs may include Microsoft Windows, Mac OS X, and Linux.

The term “processor” may include any integrated circuit or other electronic device (or collection of such electronic devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., a silicon “die”), or may be distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display may allow a user to view, enter, and/or edit information that may be relevant to the operation of the system. An input device 132, including alphanumeric and other keys, may be coupled to bus 137 for communicating information and command selections to processor 138. Another type of user input device may include cursor control 133, such as a mouse, a trackball, or cursor direction keys for communicating direction information and command selections to processor 138, and for controlling cursor movement on display 131. Such an input device may include two degrees of freedom in two axes, a first axis (e.g., x) and a second axis (e.g., y), that may allow the device to specify positions in a plane.

The computer system 140 may be used for implementing the methods and techniques described herein. According to an embodiment, those methods and techniques may be performed by computer system 140 in response to processor 138 executing one or more sequences of one or more instructions contained in main memory 134. Such instructions may be read into main memory 134 from another computer readable medium, such as storage device 135. Execution of the sequences of instructions contained in main memory 134 may cause processor 138 to perform the process steps described herein. In alternative embodiments, hard-wired circuitry may be used in place of or in combination with software instructions to implement the arrangement. Thus, embodiments of the invention may not be limited to any specific combination of hardware circuitry and software.

The term “computer readable medium” (or “machine readable medium”) as used herein is an extensible term that refers to any medium or any memory, that participates in providing instructions to a processor, (such as processor 138), for execution, or any mechanism for storing or transmitting information in a form readable by a machine (e.g., a computer). Such a medium may store computer-executable instructions to be executed by a processing element and/or control logic, and data which may be manipulated by a processing element and/or control logic, and may take many forms, including but not limited to, non-volatile medium, volatile medium, and transmission medium. Transmission media may include coaxial cables, copper wire and fiber optics, including the wires that comprise bus 137. Transmission media may also take the form of acoustic or light waves, such as those generated during radio-wave and infrared data communications, or other form of propagated signals (e.g., carrier waves, infrared signals, digital signals, etc.). Common forms of computer readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 138 for execution. For example, the instructions may initially be carried on a magnetic disk of a remote computer. The remote computer may load the instructions into its dynamic memory and send the instructions over a telephone line using a modem. A modem local to computer system 140 may receive the data on the telephone line and use an infra-red transmitter to convert the data to an infra-red signal. An infra-red detector may receive the data carried in the infra-red signal, and appropriate circuitry may place the data on bus 137. Bus 137 may carry the data to main memory 134, from which processor 138 may retrieve and execute the instructions. The instructions received by main memory 134 may optionally be stored on storage device 135 either before or after execution by processor 138.

Computer system 140 may also include a communication interface 141 coupled to bus 137. Communication interface 141 may provide a two-way data communication coupling to a network link 139 that may be connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 may typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Feb. 20, 2004), which is incorporated in its entirety for all purposes as if fully set forth herein.

Wireless links may also be implemented. In any such implementation, communication interface 141 may send and receive electrical, electromagnetic or optical signals that may carry digital data streams representing various types of information.

Network link 139 may typically provide data communication through one or more networks to other data devices. For example, network link 139 may provide a connection through local network 111 to a host computer or to data equipment operated by an Internet Service Provider (ISP) 142. ISP 142, in turn, may provide data communication services through the world wide packet data communication network Internet 11. Local network 111 and Internet 11 may both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

In view of the above, minimization and/or prevention of the occurrence of hypoglycemia through use of the AP discussed herein may be readily applicable into devices with (for example) limited processing power, such as glucose, insulin, and AP devices, and may be implemented and utilized with the related processors, networks, computer systems, internet, and components and functions according to the schemes disclosed herein.

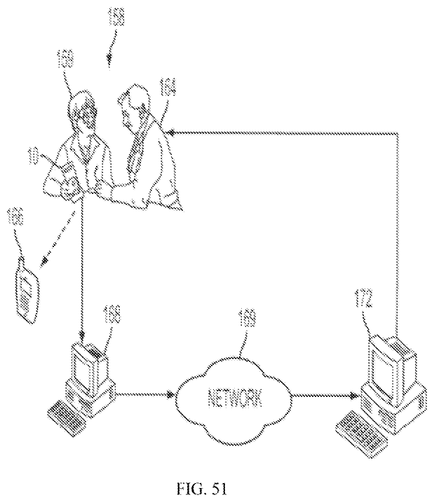

Referring to FIG. 51, there is shown an exemplary system in which examples of the invention may be implemented. In an embodiment, the CGM, the AP or the insulin device may be implemented by a subject (or patient) locally at home or at another desired location. However, in an alternative embodiment, one or more of the above may be implemented in a clinical setting. For instance, a clinical setup 158 may provide a place for doctors (e.g., 164) or clinician/assistant to diagnose patients (e.g., 159) with diseases related with glucose, and related diseases and conditions. A CGM 10 may be used to monitor and/or test the glucose levels of the patient—as a standalone device. It should be appreciated that while only one CGM 10 is shown in the figure, the system may include other AP components. The system or component, such as the CGM 10, may be affixed to the patient or in communication with the patient as desired or required. For example, the system or combination of components thereof—including a CGM 10 (or other related devices or systems such as a controller, and/or an AP, an insulin pump, or any other desired or required devices or components)—may be in contact, communication or affixed to the patient through tape or tubing (or other medical instruments or components) or may be in communication through wired or wireless connections. Such monitoring and/or testing may be short term (e.g., a clinical visit) or long term (e.g., a clinical stay). The CGM may output results that may be used by the doctor (, clinician or assistant) for appropriate actions, such as insulin injection or food feeding for the patient, or other appropriate actions or modeling. Alternatively, the CGM 10 may output results that may be delivered to computer terminal 168 for instant or future analyses. The delivery may be through cable or wireless or any other suitable medium. The CGM 10 output from the patient may also be delivered to a portable device, such as PDA 166. The CGM 10 output may also be delivered to a glucose monitoring center 172 for processing and/or analyzing. Such delivery can be accomplished in many ways, such as network connection 170, which may be wired or wireless.

In addition to the CGM 10 output, errors, parameters for accuracy improvements, and any accuracy related information may be delivered, such as to computer 168, and/or glucose monitoring center 172 for performing error analyses. Doing so may provide centralized monitoring of accuracy, modeling and/or accuracy enhancement for glucose centers, relative to assuring a reliable dependence upon glucose sensors.

Examples of the invention may also be implemented in a standalone computing device associated with the target glucose monitoring device. An exemplary computing device (or portions thereof) in which examples of the invention may be implemented is schematically illustrated in FIG. 48.

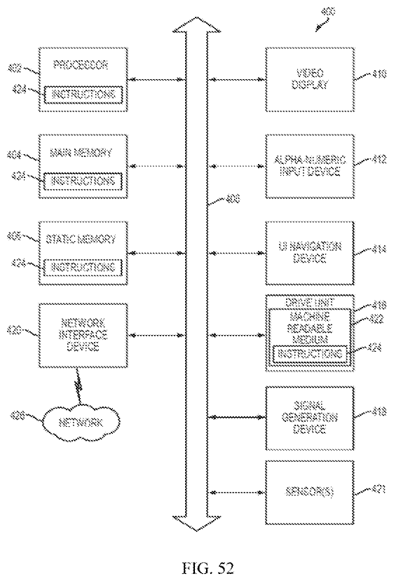

FIG. 52 provides a block diagram illustrating an exemplary machine upon which one or more aspects of embodiments, including methods thereof, herein may be implemented.

Machine 400 may include logic, one or more components, and circuits (e.g., modules). Circuits may be tangible entities configured to perform certain operations. In an example, such circuits may be arranged (e.g., internally or with respect to external entities such as other circuits) in a specified manner. In an example, one or more computer systems (e.g., a standalone, client or server computer system) or one or more hardware processors (processors) may be configured with or by software (e.g., instructions, an application portion, or an application) as a circuit that operates to perform certain operations as described herein. In an example, the software may reside (1) on a non-transitory machine readable medium or (2) in a transmission signal. In an example, the software, when executed by the underlying hardware of the circuit, may cause the circuit to perform the certain operations.

In an example, a circuit may be implemented mechanically or electronically. For example, a circuit may comprise dedicated circuitry or logic that may be specifically configured to perform one or more techniques such as are discussed above, including a special-purpose processor, a field programmable gate array (FPGA) or an application-specific integrated circuit (ASIC). In an example, a circuit may comprise programmable logic (e.g., circuitry, as encompassed within a general-purpose processor or other programmable processor) that may be temporarily configured (e.g., by software) to perform certain operations. It will be appreciated that the decision to implement a circuit mechanically (e.g., in dedicated and permanently configured circuitry), or in temporarily configured circuitry (e.g., configured by software) may be driven by cost and time considerations.

Accordingly, the term “circuit” may be understood to encompass a tangible entity, whether physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor may be configured as respective different circuits at different times. Software may accordingly configure a processor, for example, to constitute a particular circuit at one instance of time and to constitute a different circuit at a different instance of time.

In an example, circuits may provide information to, and receive information from, other circuits. In this example, the circuits may be regarded as being communicatively coupled to one or more other circuits. Where multiple of such circuits exist contemporaneously, communications may be achieved through signal transmission (e.g., over appropriate circuits and buses) that connect the circuits. In embodiments in which multiple circuits are configured or instantiated at different times, communications between such circuits may be achieved, for example, through the storage and retrieval of information in memory structures to which the multiple circuits have access. For example, one circuit may perform an operation and store the output of that operation in a memory device to which it is communicatively coupled. A further circuit may then, at a later time, access the memory device to retrieve and process the stored output. In an example, circuits may be configured to initiate or receive communications with input or output devices and may operate on a collection of information.

The various operations of methods described herein may be performed, at least partially, by one or more processors that may temporarily configured (e.g., by software) or permanently configured to perform the relevant operations. Whether temporarily or permanently configured, such processors may constitute processor-implemented circuits that operate to perform one or more operations or functions. In an example, the circuits referred to herein may comprise processor-implemented circuits.

Similarly, the methods described herein may be at least partially processor-implemented. For example, at least some of the operations of a method may be performed by one or processors or processor-implemented circuits. The performance of certain of the operations may be distributed among the one or more processors, not only residing within a single machine, but deployed across a number of machines. In an example, the processor or processors may be located in a single location (e.g., within a home environment, an office environment or as a server farm), while in other examples the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., Application Program Interfaces (APIs)).

Example embodiments (e.g., apparatus, systems, or methods) may be implemented in digital electronic circuitry, in computer hardware, in firmware, in software, or in any combination thereof. Example embodiments may be implemented using a computer program product (e.g., a computer program, tangibly embodied in an information carrier or in a machine readable medium, for execution by, or to control the operation of, data processing apparatus such as a programmable processor, a computer, or multiple computers).

A computer program may be written in any form of programming language, including compiled or interpreted languages, and may be deployed in any form, including as a stand-alone program or as a software module, subroutine, or other unit suitable for use in a computing environment. A computer program may be deployed to be executed on one computer or on multiple computers at one site or distributed across multiple sites and interconnected by a communication network.

In an example, operations may be performed by one or more programmable processors executing a computer program to perform functions by operating on input data and generating output. Examples of method operations may also be performed by, and example apparatus can be implemented as, special purpose logic circuitry (e.g., a field programmable gate array (FPGA) or an application-specific integrated circuit (ASIC)).

The computing system or systems herein may include clients and servers. A client and server may generally be remote from each other and generally interact through a communication network. The relationship of client and server arises by virtue of computer programs running on the respective computers and having a client-server relationship to each other. In embodiments deploying a programmable computing system, it will be appreciated that both hardware and software architectures may be adapted, as appropriate. Specifically, it will be appreciated that whether to implement certain functionality in permanently configured hardware (e.g., an ASIC), in temporarily configured hardware (e.g., a combination of software and a programmable processor), or a combination of permanently and temporarily configured hardware may be a function of efficiency. Below are set out hardware (e.g., machine 400) and software architectures that may be implemented in or as example embodiments.

In an example, the machine 400 may operate as a standalone device or the machine 400 may be connected (e.g., networked) to other machines.

In a networked deployment, the machine 400 may operate in the capacity of either a server or a client machine in server-client network environments. In an example, machine 400 may act as a peer machine in peer-to-peer (or other distributed) network environments. The machine 400 may be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the embodiments discussed herein.

Example machine (e.g., computer system) 400 may include a processor 402 (e.g., a central processing unit (CPU), a graphics processing unit (GPU) or both), a main memory 404 and a static memory 406, some or all of which may communicate with each other via a bus 408. The machine 400 may further include a display unit 410, an alphanumeric input device 412 (e.g., a keyboard), and a user interface (UI) navigation device 411 (e.g., a mouse). In an example, the display unit 410, input device 412 and UI navigation device 414 may be a touch screen display. The machine 400 may additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 may include a machine readable medium 422 on which is stored one or more sets of data structures or instructions 424 (e.g., software) embodying or utilized by any one or more of the methodologies or functions described herein. The instructions 424 may also reside, completely or at least partially, within the main memory 404, within static memory 406, or within the processor 402 during execution thereof by the machine 400. In an example, one or any combination of the processor 402, the main memory 404, the static memory 406, or the storage device 416 may constitute machine readable media.

While the machine readable medium 422 is illustrated as a single medium, the term “machine readable medium” may include a single medium or multiple media (e.g., a centralized or distributed database, and/or associated caches and servers) that may be configured to store the one or more instructions 424. The term “machine readable medium” may also be taken to include any tangible medium that may be capable of storing, encoding, or carrying instructions for execution by the machine and that cause the machine to perform any one or more of the embodiments of the present disclosure or that may be capable of storing, encoding or carrying data structures utilized by or associated with such instructions. The term “machine readable medium” may accordingly be understood to include, but not be limited to, solid-state memories, and optical and magnetic media. Specific examples of machine readable media may include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 may further be transmitted or received over a communications network 426 using a transmission medium via the network interface device 420 utilizing any one of a number of transfer protocols (e.g., frame relay, IP, TCP, UDP, HTTP, etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” may include any intangible medium that may be capable of storing, encoding or carrying instructions for execution by the machine, and includes digital or analog communications signals or other intangible medium to facilitate communication of such software.

Although the present embodiments have been described in detail, those skilled in the art will understand that various changes, substitutions, variations, enhancements, nuances, gradations, lesser forms, alterations, revisions, improvements and knock-offs of the embodiments disclosed herein may be made without departing from the spirit and scope of the embodiments in their broadest form.

### REFERENCES

The devices, systems, apparatuses, modules, compositions, computer program products, non-transitory computer readable medium, models, algorithms, and methods of various embodiments disclosed herein may utilize aspects (devices, systems, apparatuses, modules, compositions, computer program products, non-transitory computer readable medium, models, algorithms, and methods) disclosed in the following references, applications, publications and patents and which are hereby incorporated by reference herein in their entirety, and which are not admitted to be prior art with respect to the present embodiments by inclusion in this section:
1. Kovatchev B. A century of diabetes technology: signals, models, and artificial pancreas control. Trends Endocrinol Metab. 2019; 30(7): 432-444. PMID: 31151733
2. The Diabetes Control and Complications Trial Research Group. The effect of intensive treatment of diabetes on the development and progression of long-term complications of insulin-dependent diabetes mellitus. N Engl J Med 329: 978-986, 1993. PMID: 8366922
3. Kovatchev B P, Cox D J, Gonder-Frederick L A, W L Clarke (2002). Methods for quantifying self-monitoring blood glucose profiles exemplified by an examination of blood glucose patterns in patients with Type 1 and Type 2 Diabetes. Diabetes Technology and Therapeutics, 4 (3): 295-303. PMID: 12165168;
4. Kovatchev B P, Cox D J, Kumar A, Gonder-Frederick L A, Clarke W L (2003). Algorithmic Evaluation of Metabolic Control and Risk of Severe Hypoglycemia in Type 1 and Type 2 Diabetes Using Self-Monitoring Blood Glucose (SMBG) Data. Diabetes Technology and Therapeutics, 5 (5): 817-828. PMID: 14633347;
5. Kovatchev, B. P. Diabetes Technology: Markers, Monitoring, Assessment, and Control of Blood Glucose Fluctuations in Diabetes. Scientifica, (2012), 1-14. PMID: 24278682
6. Mastrototaro J J. The MiniMed continuous glucose monitoring system. Diabetes Technol Ther. 2000; 2: Sup 1: S13-S18. PMID: 11469627
7. Feldman B, Brazg R, Schwartz S, Weinstein R. A continuous glucose sensor based on wired enzyme technology—results from a 3-day trial in patients with type 1 diabetes. Diabetes Technol Ther. 2003; 5: 769-778. PMID: 14633342
8. Klonoff D C. Continuous glucose monitoring: roadmap for 21^{st} century diabetes therapy. Diabetes Care. 2005; 28: 1231-1239. PMID: 15855600
9. Kovatchev B P. Metrics for glycaemic control: from HbA1c to continuous glucose monitoring. Nature Reviews Endocrinology. 2017; 13: 425-436. PMID: 28304392
10. Dalla Man C, Rizza R A, and Cobelli C. Meal simulation model of the glucose-insulin system. IEEE Trans Biomed Eng, 54:1740-1749, 2007. PMID: 17926672
11. Cobelli, C., Dalla Man, C., Sparacino, G., Magni, L., Nicolao, G. & Kovatchev, B. P. Diabetes: Models, Signals, and Control. IEEE Reviews in Biomedical Engineering (2009) 2:54-96. PMID: 20936056; PMCID: PMC2951686
12. Kovatchev, B. P., Breton, M. D., Dalla Man, C. & Cobelli, C. In Silico Preclinical Trials: A Proof of Concept in Closed-Loop Control of Type 1 Diabetes. J. Diabetes Sci. Technol. 3, 44-55 (2009). PMID: 19444330, PMCID: PMC2681269
13. Dalla Man, C., Micheletto, F., Lv, D., Breton, M., Kovatchev, B., & Cobelli, C. (2014). The UVA/PADOVA Type 1 Diabetes Simulator New Features. J Diabetes Sci Technology, 8(1), 26-34. PMID: 24876534
14. Viceconti M, Cobelli C, Haddad T, Himes A, Kovatchev B P, Palmer M. In silico assessment of biomedical products: The conundrum of rare but not so rare events in two case studies. J Engineering in Medicine 2017, 231: 455-466. PMID: 28427321
15. Friedrich MJ. Artificial pancreas may soon be a reality. JAMA. 2009; 301: 1525-1527. PMID: 19366764
16. Dolgin E. Managed by machine. Nature. 2012; 485: S6-S8. PMID: 22616096
17. Clery D. A pancreas in a box. Science. 2014; 343:133-15. PMID: 24408414
18. Cobelli C., Renard E. and Kovatchev B. P. The artificial pancreas: a digital-age treatment for diabetes. Lancet Diabetes & Endocrinology, doi:10.1016/S2213-8587(14)70126-3, 2014. PMID: 24943066
19. Kovatchev B P, Renard E, Cobelli C, Zisser H, Keith-Hynes P, Anderson S M, Brown S A, Chernavvsky D R, Breton M D, Farret A, Pelletier M, Place J, Bruttomesso D, Del Favero S, Visentin R, Filippi A, Scotton R, Avogaro A, Doyle F III. Feasibility of outpatient fully integrated closed-loop control: first studies of wearable artificial pancreas. Diabetes Care. 2013; 36: 1851-1858. PMCID: PMC3687268
20. Bergenstal, R. M. et al. Safety of a Hybrid Closed-Loop Insulin Delivery System in Patients With Type 1 Diabetes. JAMA Published online Sep. 15, 2016. PMID: 27629148
21. Kovatchev B P, Anderson S M, Raghinaru D, et al, for the iDCL Trial Research Group. Randomized Controlled Trial of Mobile Closed-Loop Control. Diabetes Care. 2020; 43:607-615. PMID: 31937608
22. Brown S A, Kovatchev B P, Raghinaru D, et al, for the iDCL Trial Research Group. 6-month randomized multicenter trial of closed-loop control in type 1 diabetes. N Engl J Med. 2019; 381: 1707-17. PMID: 31618560.
23. Breton M D, Kanapka L G, Beck R W, et al, for the iDCL Trial Research Group. A Randomized Trial of Closed-Loop Control in Children with Type 1 Diabetes. N Engl J Med. 2020; 383:836-845. PMID: 32846062.
24. UK Prospective Diabetes Study Group (UKPDS). Intensive blood-glucose control with sulphonylureas or insulin compared with conventional treatment and risk of complications in patients with type 2 diabetes. Lancet 352: 837-853, 1998. PMID: 9742976
25. Nauck M A, Homberger E, Siegel E G, Allen R C, Eaton R P, Ebert R, Creutzfeldt W. 1986. Incretin effects of increasing glucose loads in man calculated from venous insulin and C-peptide responses. J Clin Endocrinol Metab. 63:492-8.
26. Nauck M, Stockmann F, Ebert R, Creutzfeldt W. 1986. Reduced incretin effect in type 2 (non-insulin-dependent) diabetes. Diabetologia 29:46-52.
27. Drucker D J, Nauck M A. 2006. The incretin system: glucagon-like peptide-1 receptor agonists and dipeptidyl peptidase-4 inhibitors in type 2 diabetes. Lancet 368:1696-1705.
28. Van Cauter E, Fabienne Mestrez, Jeppe Sturis and Kenneth S Polonsky, ‘Estimation of insulin secretion rates from C-peptide levels: Comparison of individual and standard kinetic parameters for C-peptide clearance’, Diabetes, 41(3):368-377, 1992
29. Matthews D R, J. P. Hosker, A. S. Rudenski, B. A. Naylor, D. F. Treacher & R. C. Turner; Homeostasis model assessment: insulin resistance and β-cell function from fasting plasma glucose and insulin concentrations in man. Diabetologia (1985) 28:412-419
30. Ahlqvist E, P. Storm, A. Käräjämäki, M. Martinell, M. Dorkhan, A. Carlsson, et al., ‘Novemberel subgroups of adult-onset diabetes and their association with outcomes: A data-driven cluster analysis of six variables’, Lancet Diabetes Endocinol, 6:361-369, 2018

What is claimed is:

1. A method of mapping a metabolic phenotype, comprising:
obtaining one or more in vivo glucose-metabolism traits of a subject;
comparing the one or more in vivo glucose-metabolism traits of the subject to a corresponding one or more glucose-metabolism traits of one or more in silico entities;
based on the comparing, determining at least one matching in silico entity for the one or more glucose-metabolism traits of the subject; and
assigning in vivo behavioral and demographic characteristics for the subject to the at least one matching in silico entity.

2. The method of claim 1, wherein:
said glucose-metabolism traits comprise one or more of (a) hemoglobin A1c (HbA1c), (b) fasting glucose, (c) C-peptide, (d) HOMA2-B, (e) HOMA-IR, or (f) any combination thereof.

3. The method of claim 1, wherein:
said behavioral and demographic characteristics comprise one or more of (1) age of the subject, (m) duration of diabetes for the subject, (n) body mass index (BMI) of the subject, (o) body weight (BW) of the subject, or (p) any combination thereof.

4. The method of claim 1, wherein:
the determining and/or the assigning are performed, depending upon the availability of data therefor, in a single pass or iteratively.

5. The method of claim 1, wherein:
the subject comprises a human or an animal, relative to a number of corresponding images of an in silico entity therefor as stored in a database comprising a population of in silico entity images.

6. The method of claim 1, wherein:
the determining at least one matching in silico entity for the one or more glucose-metabolism traits of the subject is based on a least magnitude Euclidean distance evaluated for at least (x) the subject and the at least one matching in silico entity for the one or more glucose-metabolism traits of the subject and (y) the subject and another in silico entity for the one or more glucose-metabolism traits of the subject.

7. A system for mapping a metabolic phenotype, comprising:
a processor;
a processor-readable memory including processor-executable instructions for:
obtaining one or more in vivo glucose-metabolism traits of a subject;
comparing the one or more in vivo glucose-metabolism traits of the subject to a corresponding one or more glucose-metabolism traits of one or more in silico entities;
based on the comparing, determining at least one matching in silico entity for the one or more glucose-metabolism traits of the subject; and
assigning in vivo behavioral and demographic characteristics for the subject to the at least one matching in silico entity.

8. The system of claim 7, wherein:
said glucose-metabolism traits comprise one or more of (a) hemoglobin A1c (HbA1c), (b) fasting glucose, (c) C-peptide, (d) HOMA2-B, (e) HOMA-IR, or (f) any combination thereof.

9. The system of claim 7, wherein:
said behavioral and demographic characteristics comprise one or more of (1) age of the subject, (m) duration of diabetes for the subject, (n) body mass index (BMI) of the subject, (o) body weight (BW) of the subject, or (p) any combination thereof.

10. The system of claim 7, wherein:
the determining and/or the assigning are performed, depending upon the availability of data therefor, in a single pass or iteratively.

11. The system of claim 7, wherein:
the subject comprises a human or an animal, relative to a number of corresponding images of an in silico entity therefor as stored in a database comprising a population of in silico entity images.

12. The system of claim 7, wherein:
the determining at least one matching in silico entity for the one or more glucose-metabolism traits of the subject is based on a least magnitude Euclidean distance evaluated for at least (x) the subject and the at least one matching in silico entity for the one or more glucose-metabolism traits of the subject and (y) the subject and another in silico entity for the one or more glucose-metabolism traits of the subject.

13. A non-transient computer-readable medium having stored thereon computer-readable instructions for mapping a metabolic phenotype, said instructions comprising instructions causing a computer to:
receive one or more in vivo glucose-metabolism traits of a subject;
compare the one or more in vivo glucose-metabolism traits of the subject to a corresponding one or more glucose-metabolism traits of one or more in silico entities;
based on the comparison, determine at least one matching in silico entity for the one or more glucose-metabolism traits of the subject; and
assign in vivo behavioral and demographic characteristics for the subject to the at least one matching in silico entity.

14. The medium of claim 13, wherein:
said glucose-metabolism traits comprise one or more of (a) hemoglobin A1c (HbA1c), (b) fasting glucose, (c) C-peptide, (d) HOMA2-B, (e) HOMA-IR, or (f) any combination thereof.

15. The medium of claim 13, wherein:
said behavioral and demographic characteristics comprise one or more of (l) age of the subject, (m) duration of diabetes for the subject, (n) body mass index (BMI) of the subject, (o) body weight (BW) of the subject, or (p) any combination thereof.

16. The medium of claim 13, wherein:
the determining and/or the assigning are performed, depending upon the availability of data therefor, in a single pass or iteratively.

17. The medium of claim 13, wherein:
the subject comprises a human or an animal, relative to a number of corresponding images of an in silico entity therefor as stored in a database comprising a population of in silico entity images.

18. The medium of claim 13, wherein:
the determining at least one matching in silico entity for the one or more glucose-metabolism traits of the subject is based on a least magnitude Euclidean distance evaluated for at least (x) the subject and the at least one matching in silico entity for the one or more glucose-metabolism traits of the subject and (y) the subject and another in silico entity for the one or more glucose-metabolism traits of the subject.
